(19) United States
(12) Patent Application Publication
(10) Pub. No.: US 2025/0135172 A1
(43) Pub. Date: May 1, 2025

(54) NEEDLE ROLLER DEVICE FOR SKIN INJECTION

(71) Applicants: Hyun Mee NOH, Yongin-si (KR); NOBAMEDI CO., LTD., Yongin-si (KR)

(72) Inventor: Hyun Mee NOH, Yongin-si (KR)

(73) Assignees: Hyun Mee NOH, Yongin-si (KR); NOBAMEDI CO., LTD., Yongin-si (KR)

(21) Appl. No.: 18/666,422

(22) Filed: May 16, 2024

(30) Foreign Application Priority Data
Oct. 31, 2023 (KR) 10-2023-0148305

(51) Int. Cl. A61M 37/00

(52) U.S. Cl. CPC A61M 37/00

(57) ABSTRACT

Provided is a needle roller device for skin injection which includes a main body comprising, formed on one side thereof, an injection pipe part with an injection port to which a drug injection device is detachably coupled, and formed on the other side thereof, an expansion part with predetermined width and length communicating with the injection pipe part, a roller part rotatably coupled to the expansion part, and comprising a plurality of needle protrusions formed on an outer circumferential surface, and fine needles coupled to the plurality of needle protrusions and exposed to outside, and a rotation connection part that fastens the main body and the roller part.

### CROSS-REFERENCE TO RELATED APPLICATION

This application claims priority to Korean Patent Applications No. 10-2023-0148305, filed in the Korean Intellectual Property Office on Oct. 31, 2023, the entire contents of which are hereby incorporated by reference.

### BACKGROUND

### Technical Field

The present disclosure relates to a needle roller device for skin injection, and more specifically, to a needle roller device for skin injection that utilizes microscopic needles to form micropores on the surface of the skin, thereby stimulating the skin and aiding in the regeneration of the skin.

### Description of Related Art

In general, wrinkles are related to corium that constitutes the bottom layer of the epidermis, and is composed of a layer several times thicker than the epidermis, with a thickness of approximately 0.7 mm to 4 mm.

The amount of collagen in the corium gradually decreases as aging progresses, resulting in dry skin and increased wrinkles. It is necessary to continuously supplement the consumed collagen or inject new collagen to prevent wrinkles or maintain elastic skin.

In the related art, this involves a method of applying or spraying nutrients such as vitamin C and peptides necessary for collagen formation to the skin to prevent or improve wrinkles or to make skin elastic.

When using this method, the possibility of the contents that can pass through the skin's stratum corneum is only 0.3% of the total content, and the remaining 99.7% do not pass through the sebum on the outermost part of the skin and remain on the skin or dry out and disappear. Therefore, there is a limit to improving wrinkles or giving elasticity to the skin by using gel, lotion, or the like.

To improve this, a needle roller has been proposed, which not only stimulates the skin using a fine needle roller, but also allows the fine needles exposed on the outer surface of the roller to create microchannels in the skin to supply nutrients necessary for collagen formation, such as vitamin C and peptides, to the dermal layer.

This related art includes a handle and a roller, and fine needles radially formed on an outer surface of a cylindrical roller.

Therefore, by moving the roller against the skin, the roller is rotated to form a number of micropores in the skin, and the drug applied to the skin can be absorbed into the skin through the micropores formed in this way.

Meanwhile, in the related art, the fine needles are provided in the form of a gear that forms a micropore in the skin with each tooth, but there are problems in that the outer surface of the roller rubs against the skin, making it difficult to insert the fine needles effectively into the skin, and even if the needles are inserted, the fine needles causes skin damage by being pushed by the roller, leaving scars, and increasing the risk of pain and infection.

In addition, in the related art, there is a problem that the drug may not be applied to the fine needles during drug application process, if the fine needles are damaged or the fine needles are spaced far from the drug supply pipe.

### SUMMARY

The present disclosure was devised to solve the problems described above, and an object of the present disclosure is to provide a needle roller device for skin injection, which is capable of minimizing damage to the skin in the process of moving a roller with fine needles formed thereon in close contact with the skin, by allowing the fine needles to penetrate the skin and form micropores in the skin, and also capable of allowing nutrients in a drug to be effectively absorbed into the skin using the micropores.

In addition, another object of the present disclosure is to provide a needle roller device for skin injection, which is capable of allowing the drug to be applied to the fine needles without damaging the fine needles.

Yet another object of the present disclosure is to provide a needle roller device for skin injection, which is capable of allowing simple and solid fastening and also reducing friction caused by the rotation of the roller, thereby enabling a smooth procedure.

An object of the present disclosure may be achieved by a needle roller device for skin injection, which may include a main body comprising, formed on one side thereof, an injection pipe part with an injection port to which a drug injection device is detachably coupled, and formed on the other side thereof, an expansion part with predetermined width and length communicating with the injection pipe part, a roller part rotatably coupled to the expansion part, and comprising a plurality of needle protrusions formed on an outer circumferential surface, and fine needles coupled to the plurality of needle protrusions and exposed to outside, and a rotation connection part that fastens the main body and the roller part.

The main body may include an injection passage formed therein, the expansion part may include a plurality of distribution passages communicating with the injection passage and divided into a plurality of distribution passages, and a plurality of distribution nozzles with each distribution passage formed therein.

The distribution nozzle may include a cut groove formed at a tip such that the needle passes therethrough. The cut groove may be recessed inwardly into the distribution nozzle, and an end of the fine needle is introduced into the distribution nozzle, while being spaced apart from an end of the cut groove.

The expansion part may include an outlet formed on an outer surface, and a drain passage connected to the outlet and communicating with the injection passage.

The roller part may include a plurality of connected needle blocks, the needle block may be formed by coupling first and second disk covers and fine needle disks positioned between the disk covers to each other, the first and second disk covers may each include a plurality of needle protrusions protruding from an outer circumferential surface, the fine needle disk may include a plurality of fine needles formed radially on an outer circumferential surface, and the fine needles may be configured to be inserted into each needle protrusion, with an end of the fine needle protruding out of the needle protrusion.

The needle protrusion may include skin pressing parts formed at tips thereof.

The first and second disk covers may include a central through hole formed in a center of an inner surface, a pin protruding from one side of the central through hole, and a pin hole formed on the other side of the central through hole, the fine needle disk may include a plurality of fine needles radially formed on an outer circumferential surface, a first through hole formed in a center, and second and third through holes formed on both sides of the first through hole, respectively, the first and second disk covers may be coupled to each other with the fine needle disk inserted therebetween, while the pins are passed through the second and third through holes, respectively, and upon coupling the first and second disk covers and the fine needle disk positioned therebetween, the fine needles may be exposed only to the outside of the needle protrusions.

The first and second disk covers may include fine needle insertion grooves which are recessed inward and into which the fine needle disk and the fine needle are inserted.

The rotation connection part may include a rod-shaped shaft part, a first round jaw protruding from an outer surface of the shaft part, a second round jaw protruding apart from the first round jaw to be caught and fixed on an outer circumferential surface of a needle block, and a stopper formed at an end of the shaft part and fitted into a through hole of a hinge bracket.

According to some examples of the present disclosure, in the process of moving the roller with the fine needles in close contact with the skin, only the end of the needle protrusion comes into contact with the skin such that the contact area is minimized to prevent skin from being pushed back, the fine needles penetrate the skin to form micropores in the skin to minimize damage to the skin, and nutrients in the drug can be easily absorbed through the micropores formed in the skin, thereby ensuring effective regeneration of the collagen in the dermis layer.

According to some examples of the present disclosure, the drug can be applied to the fine needles without damaging the fine needles, the needle roller for skin injection can be simply and firmly fastened, and the friction caused by the rotation of the roller can be reduced, thereby allowing a smooth procedure.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1 is a perspective view of a needle roller device for skin injection according to an example of the present disclosure;

FIG. 2 is an exploded perspective view of the needle roller device for skin injection according to the example of the present disclosure;

FIG. 3 is a side cross-sectional view of the needle roller device for skin injection according to the example of the present disclosure;

FIG. 4 is a plan cross-sectional view of the needle roller device for skin injection according to the example of the present disclosure; and

FIG. 5 is a front cross-sectional view of the needle roller device for skin injection according to the example of the present disclosure.

### DETAILED DESCRIPTION

Hereinafter, certain preferred examples of the disclosure will be described in detail below based on the attached drawings.

The examples to be described below are intended to explain the present disclosure in detail such that a person skilled in the art can easily practice the present disclosure, and do not mean that the technical idea and scope of the disclosure are limited.

In addition, it is to be noted that the size or shape of the components shown in the drawings may be exaggerated for clarity and convenience of explanation, and terms specifically defined in consideration of the configuration and operation of the present disclosure may vary according to the intention or custom of the user or operator, and these terms should be defined based on the content throughout this specification.

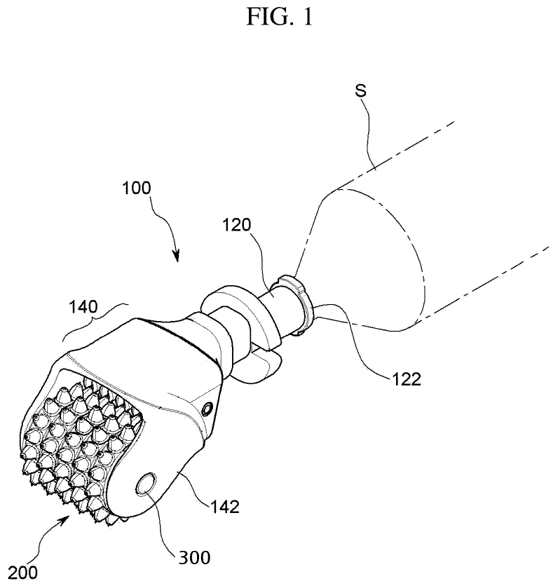

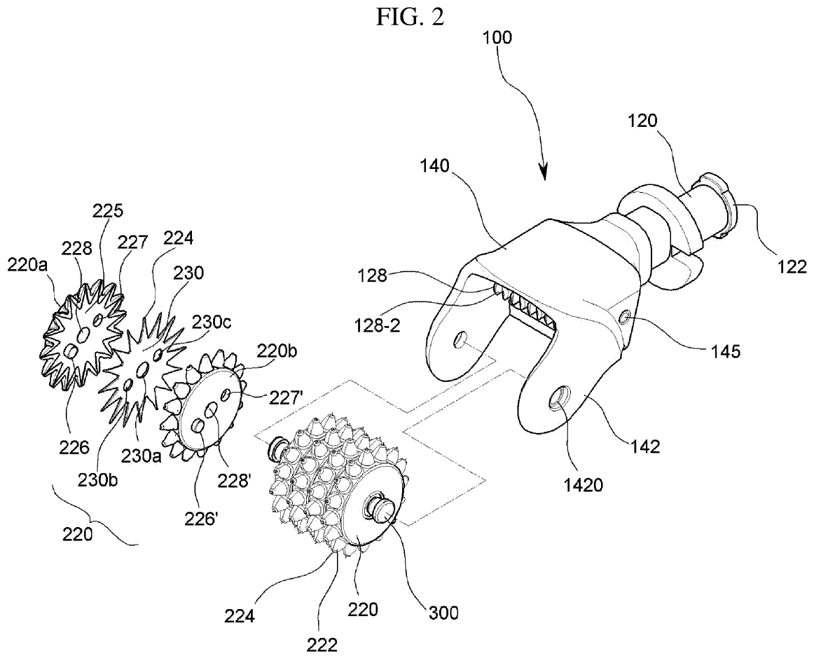

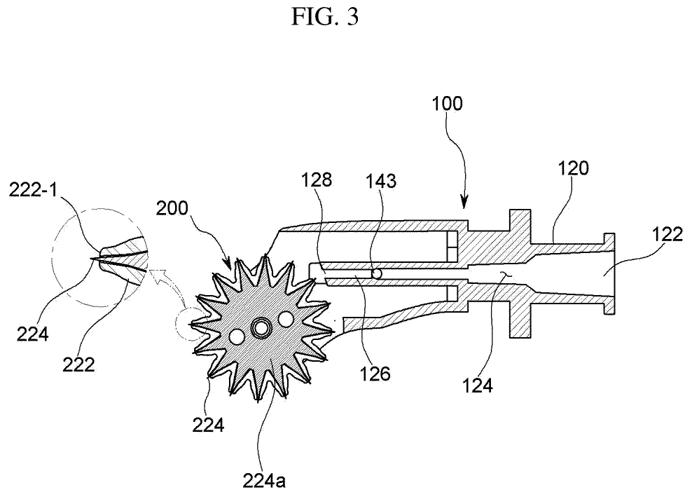

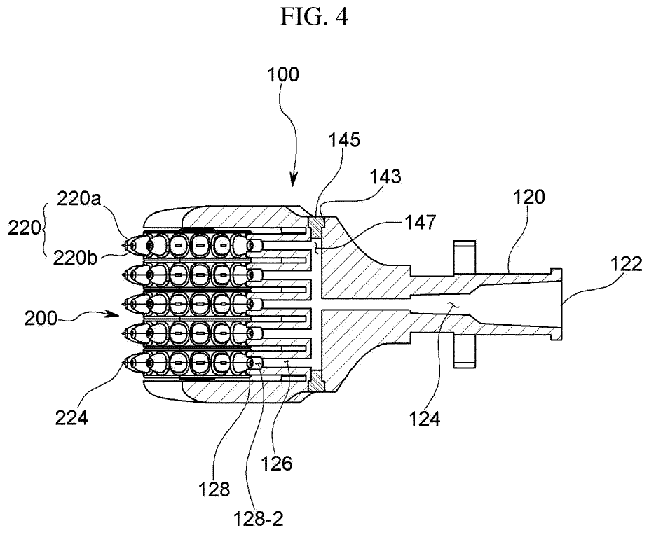

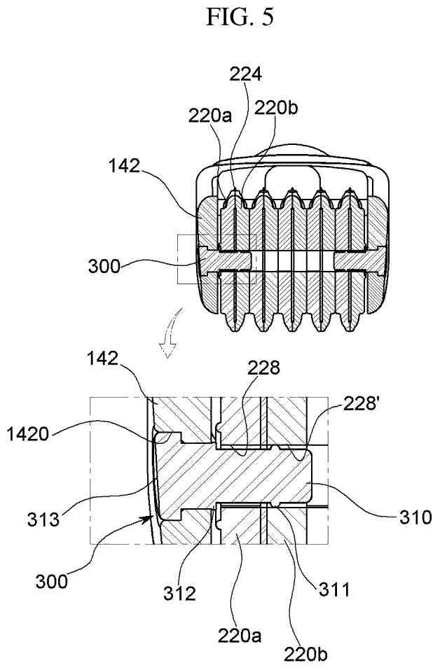

In the accompanying drawings, FIG. 1 is a perspective view of a needle roller device for skin injection according to an example of the present disclosure, FIG. 2 is an exploded perspective view of the needle roller device for skin injection according to the example of the present disclosure, FIG. 3 is an exploded cross-sectional view of the needle roller device for skin injection according to the example of the present disclosure, FIG. 4 is a plan cross-sectional view of the needle roller device for skin injection according to the example of the present disclosure, and FIG. 5 is a front cross-sectional view of the needle roller device for skin injection according to the example of the present disclosure.

As illustrated in FIGS. 1 to 5, the needle roller device for skin injection according to the example of the present disclosure includes a main body 100, a roller part 200, and a rotation connection part 300 that fastens the main body 100 with the roller part 200. A drug injection device S is coupled to the main body 100.

The drug injection device S may be an injector that automatically injects the drug or a syringe to manually inject the drug.

The main body 100 is in an approximately T shape, and includes, formed on one side, an injection pipe part 120 with an injection port 122 to which the drug injection device S is detachably coupled, and formed on the other side, an expansion part 140 having predetermined width and length and communicating with the injection pipe part 120.

The expansion part 140 includes hinge brackets 142 formed on both sides, and is fastened to the hinge brackets 142 by both sides of the roller part 200 and the rotation connection parts 300, respectively, so that the roller part 200 may be rotated.

The main body 100 includes an injection passage 124 formed therein, and the expansion part 140 includes a plurality of distribution passages 126 communicating with the injection passage 124 and divided into a plurality of passages, and a plurality of distribution nozzles 128 each having the distribution passage 126 formed therein.

The distribution nozzle 128 includes a cut groove 128-2 formed at a tip to allow a fine needle 232 to pass through, in which the cut groove 128-2 is formed inwardly into the distribution nozzle 128.

The drug injected through the injection pipe part 120 is injected through the injection passage 124 and then uniformly distributed by the plurality of distribution passages 126, and thus can be simultaneously ejected from the plurality of distribution nozzles 128.

An end of the fine needle 232 is introduced into the distribution nozzle 128, but is spaced apart from the end of the cut groove 128-2, thereby preventing damage to the fine needle 232 and ensuring that the drug is applied to the end of the fine needle 232.

In addition, the expansion part 140 includes an outlet 143 formed on an outer surface, and a drain passage 147 connected to the outlet 143 and communicating with the injection passage 124. The outlet 143 is coupled to a cap 145 and sealed.

Therefore, when the injection passage 124 or the distribution passage 126 is filled with bubbles, the transfer of the drug is blocked. Accordingly, the cap 145 is opened to discharge a certain amount of the drug through the outlet 143, and then the cap 145 is closed and the injection operation is performed, so that stable drug injection can be achieved.

The roller part 200 has a cylindrical shape, is rotatably coupled to the expansion part 140, and includes a plurality of needle protrusions 222 formed radially on an outer circumferential surface. The fine needles 232 are formed in the plurality of needle protrusions 222, while being exposed to the outside.

More specifically, the roller part 200 includes a plurality of needle blocks 220 connected to each other, and each of the needle blocks 220 includes the plurality of needle protrusions 222 formed on the outer circumferential surface.

The plurality of needle blocks 220 may be adjacent to each other, and the plurality of needle blocks 220 may be integrally coupled with each other by fitting pins 226 into pin holes 227, respectively.

The needle protrusion 222 protrudes from an outer circumferential surface of the needle block 220, with its bottom being integrally formed on an outer circumferential surface of the needle block 220, and decreases in diameter toward the top to form a cone shape.

In particular, a skin pressing part 222-1 is formed flat on an upper end of the needle protrusion 222 to be brought into close contact with the skin and pressed.

The needle block 220 is formed by coupling first and second disk covers 220a and 220b with a fine needle disk 230 positioned therebetween.

The first and second disk covers 220a and 220b include a plurality of needle protrusions 222 protruding from outer circumferential surfaces, and fine needle insertion grooves 225 which are recessed inward and into which the fine needle disk 230 and the fine needles 232 are inserted

The first disk cover 220a includes a central through hole 228 formed at a center of an inner surface, a pin 226 protruding from one side of the central through hole 228, and a pin hole 227 formed on the other side of the central through hole 228.

The second disk cover 220b includes a central through hole 228′ formed at a center of an inner surface, a pin 226′ protruding from one side of the central through hole 228′, and a pin hole 227′ formed on the other side of the central through hole 228′.

The pin holes 227 and 227′ are recessed in the other sides of the pins 226 and 226′ of the first and second disk covers 220a and 220b, respectively.

Therefore, upon coupling the first and second disk covers 220a and 220b to each other, the pins 226 and 226′ are fitted into the pin holes 227 and 227′, respectively.

Meanwhile, for the first and second disk covers 220a and 220b positioned on the outermost sides adjacent to the hinge brackets 142 of the expansion part 140, the pins protruding on the outer surface may be omitted, and the first and second disk covers 220a and 220b may be firmly fastened to the hinge brackets 142 by the rotation connection parts 300 and rotated.

The fine needle disk 230 includes a plurality of fine needles 232 formed radially on the outer circumferential surface, a first through hole 230a formed in the center, and second and third through holes 230b and 230c formed on both sides of the first through hole 230a, respectively.

The first through hole 230a coincides with the central through hole 228 of the needle block 220, and the second and third through holes 230b and 230c coincide with the pin 226 and the pin hole 227.

Accordingly, in order to couple the first and second disk covers 220a and 220b to each other, the fine needle disk 230 is inserted between the first disk cover 220a and the second disk cover 220b, and the first and second disk covers 220a and 220b are coupled to each other by the pins 226 passed through the second and third through holes 230b and 230c, respectively.

The fine needle disk 230 is inserted into the fine needle insertion groove 225 of the needle block 220, and each fine needle 232 is inserted into each needle protrusion 222, with only the end of the fine needle 232 of the fine needle disk 230 being exposed to the outside of the needle protrusion 222.

As a result, the plurality of needle blocks 220 are coupled adjacent to each other to form a cylindrical shape, and coupled to both hinge brackets 142 of the expansion part 140 of the main body 100 with the rotation connection parts 300 and assembled.

The rotation connection part 300 includes a rod-shaped shaft part 310, a first round jaw 311 protruding from an outer surface of the shaft part 310, a second round jaw 312 protruding apart from the first round jaw 311 to be caught and fixed on the outer circumferential surface of the needle block 220, and a stopper 313 formed at an end of the shaft part 310 and fitted into a through hole 1420 of the hinge bracket 142.

The first round jaw 311 may be fitted into the central through hole 228′ of the second disk cover 220b to stably support rotational driving and reduce friction, thereby allowing a smooth procedure.

The second round jaw 312 is closely coupled to an entrance of the central through hole 228 of the first disk cover 220a, and the stopper is fitted into the through hole of the hinge bracket 142 to provide a secondary support such that the rotation connection parts 300 on both sides may support the rotational driving and maintain stable rolling driving when the roller part 200 is rotated, thereby improving the stability of the procedure.

Then, the drug injection device S is coupled to the injection port 122 of the injection pipe part 120 of the main body 100, and the drug is injected to be ejected through the distribution nozzle 128, and as the roller part 200 is moved and rotated while in close contact with the skin, the tip of the needle protrusion 222 comes into point contact with the skin such that the fine needles 232 accurately pierce the skin and form micropores, while the skin pressing part 222-1 presses the skin and minimize skin being pushed back.

The drug applied to the skin can be absorbed into the skin through the micropores formed in the skin as described above, and skin improvement procedures can be performed in the process the micropores are regenerated.

Although the description has been made in relation to certain embodiments, it will be readily apparent to those skilled in the art that various modifications and variations can be made without departing from the gist and scope of the disclosure, and it is obvious that all such changes and modifications fall within the scope of the appended claims.

1. A needle roller device for skin injection, comprising:
a main body comprising, formed on one side thereof, an injection pipe part with an injection port to which a drug injection device is detachably coupled, and formed on the other side thereof, an expansion part with predetermined width and length communicating with the injection pipe part;
a roller part rotatably coupled to the expansion part, and comprising a plurality of needle protrusions formed on an outer circumferential surface, and fine needles coupled to the plurality of needle protrusions and exposed to outside; and
a rotation connection part that fastens the main body and the roller part.

2. The needle roller device for skin injection according to claim 1, wherein the main body includes an injection passage formed therein,
the expansion part includes a plurality of distribution passages communicating with the injection passage, and
each distribution passage includes a distribution nozzle formed at an end thereof.

3. The needle roller device for skin injection according to claim 2, wherein the distribution nozzle includes a cut groove formed at a tip.

4. The needle roller device for skin injection according to claim 1, wherein the expansion part includes an outlet on an outer surface, and
a drain passage connecting the outlet and an injection passage.

5. The needle roller device for skin injection according to claim 1, wherein the roller part includes a plurality of connected needle blocks,
the needle block is formed by coupling first and second disk covers and fine needle disks positioned between the disk covers to each other,
the first and second disk covers include a plurality of needle protrusions protruding from an outer circumferential surface,
the fine needle disk includes a plurality of fine needles formed radially on an outer circumferential surface, and
the fine needles are configured to be inserted into each needle protrusion, with an end of the fine needle protruding out of the needle protrusion.

6. The needle roller device for skin injection according to claim 1, wherein the needle protrusions include skin pressing parts formed at tips thereof.

7. The needle roller device for skin injection according to claim 5, the first and second disk covers each include a central through hole formed in a center of an inner surface, a pin protruding from one side of the central through hole, and a pin hole formed on the other side of the central through hole,
the fine needle disk includes a plurality of fine needles radially formed on an outer circumferential surface, a first through hole formed in a center, and second and third through holes formed on both sides of the first through hole, respectively,
the first and second disk covers are coupled to each other with the fine needle disk inserted therebetween, while the pins are passed through the second and third through holes, respectively, and
upon coupling the first and second disk covers and the fine needle disk positioned therebetween, the fine needles are exposed only to the outside of the needle protrusions.

8. The needle roller device for skin injection according to claim 5, wherein the first and second disk covers include fine needle insertion grooves which are recessed inward and into which the fine needle disk and the fine needle are inserted.

9. The needle roller device for skin injection according to claim 1, wherein the rotation connection part includes a rod-shaped shaft part, a first round jaw protruding from an outer surface of the shaft part, a second round jaw protruding apart from the first round jaw to be caught and fixed on an outer circumferential surface of a needle block, and a stopper formed at an end of the shaft part and fitted into a through hole of a hinge bracket.

10. The needle roller device for skin injection according to claim 9, wherein the first round jaw is fitted into a central through hole of the second disk cover,
the second round jaw is closely coupled to an entrance of a central through hole of the first disk cover, and
the stopper is fitted into the through hole of the hinge bracket such that the rotation connection parts on both sides support rotation driving when the roller part is rotated.

11. The needle roller device for skin injection according to claim 3, wherein the cut groove is recessed inwardly into the distribution nozzle, and
an end of the fine needle is introduced into the distribution nozzle, while being spaced apart from an end of the cut groove.
